(19) United States
(12) Patent Application Publication
(10) Pub. No.: US 2023/0218800 A1
(43) Pub. Date: Jul. 13, 2023

(54) THREE-DIMENSIONAL NETWORK AQUEOUS GEL AND MANUFACTURING METHOD THEREOF

(71) Applicant: Nai-Wen MEI, Taipei City (TW)

(72) Inventors: Nai-Wen MEI, Taipei City (TW); Hui-Ching MEI, New Taipei City (TW)

(21) Appl. No.: 18/095,538

(22) Filed: Jan. 11, 2023

(30) Foreign Application Priority Data
Jan. 11, 2022 (TW) 111101154

(51) Int. Cl. A61L 26/00; C08J 9/00

(52) U.S. Cl. CPC A61L 26/0052; A61L 26/0085; A61L 26/008; A61L 26/0023; A61L 26/0014; A61L 26/009; C08J 9/0061; C08J 2205/044; A61L 2430/34; C08J 2207/10; C08J 2301/28; C08J 2405/04; C08J 2439/06

(57) ABSTRACT

A three-dimensional network aqueous gel and a manufacturing method thereof are disclosed. A water-soluble polymer is first added into a solvent and uniformly mixed, followed by hydrolysis to form a sol, and vacuum is applied to convert the sol into a gel, followed by a polycondensation reaction to form a three-dimensional network aqueous gel. The three-dimensional network aqueous gel is formed of the water-soluble polymer that includes a group including sodium alginate and sodium carboxymethyl cellulose. The water-soluble polymer is interconnected to form a three-dimensional network structure. The three-dimensional network aqueous gel is of a gel-enclosed form, which uses the three-dimensional network structure formed of a high-molecule polymer to enclose medicine, so as to more effectively protect the active ingredient and provide an effect of controlled released to thereby extend therapeutic period and reduce side effects of irritating skin.

### TECHNICAL FIELD OF THE INVENTION

The present invention relates to a three-dimensional network aqueous gel, and a manufacturing method thereof, and more particularly to a three-dimensional network aqueous gel that speeds up healing of wounds of all sorts and prevents abnormal healing of wounds, and a manufacturing method thereof.

### DESCRIPTION OF THE PRIOR ART

Skin is the largest organ of a human body and has functions of protecting against invasion of external bacteria, regulating balance of body liquid, and sensing external excitations. When a would appears on the skin, the skin is no longer capable of the function of protecting. The wound, if not properly handled, may lead to infection or inflammation. Thus, the wound must be handled in a correct way to avoid wound infection. It is a common practice to use an addressing having functions of hemostasis, protection, and infection prevention to cover the wound. Further, the addressing can increase the rate of skin repair and regeneration, and thus, the solution for accelerating wound healing is also a vital issue.

The dressings that are contemporarily used to cover wounds are classified as dry dressings and wet dressings. The dry dressings include gauze, and the wet dressings include film dressings, hydrophilic dressings, hydrophilic fibrous dressings, foam dressings, antimicrobial dressings, negative-pressure wound dressings, advanced therapy dressings, and active dressings. The dry dressings have the following defects: poor healing environment, easy localized dehydration of wound site, formation of scab, and wound pain caused by scabs, and consequently, loss of bioactivity, slow healing rate, and frequent replacement of dressings for fast leakage. Further, the dressing may get adhered to newly growing granulation tissue so that the wound would be damaged in replacing the dressings, and in addition, there is no isolative barrier from the outside, making the chance of cross infection increased.

In view of the above, the present inventor has devoted to research and study of products for wounds, hemostasis, and antiadhesion in order to enhance quality of medical care and also to provide reliable and convenient biomedical products. The present inventor has spent a lot of energy and spirit in research and study and makes breakthroughs and continuous innovation in the field to develop novel technical solutions to alleviate the defects of the prior art and to bring the society benign products and also to enhance the development of industry.

### SUMMARY OF THE INVENTION

The primary objective of the present invention is to provide a three-dimensional network aqueous gel and a manufacturing method thereof, wherein the three-dimensional network aqueous gel has excellent biocompatibility and biodegradability and is easy to synthesize, and exhibits excellent permeability for low molecular solute. Further, the three-dimensional network aqueous gel has effects of antiinflammation reaction and wound smoothness so as to reduce the formation of scabs, prevent abnormal healing of wounds, and achieve the purpose of speeding up wound healing.

To achieve the above objective, the present invention provides a three-dimensional network aqueous gel, which is formed of a water-soluble polymer, the water-soluble polymer comprises sodium carboxymethyl cellulose, the water-soluble polymer being interconnected with a solvent to form a three-dimensional network structure.

In the three-dimensional network aqueous gel according to the present invention, the water-soluble polymer further comprises sodium alginate, wherein sodium carboxymethyl cellulose and sodium alginate are interconnected with the solvent to form the three-dimensional network structure.

In the three-dimensional network aqueous gel according to the present invention, the water-soluble polymer further comprises polyvinylpyrrolidone, wherein sodium carboxymethyl cellulose, sodium alginate, and polyvinylpyrrolidone are interconnected with the solvent to form the three-dimensional network structure.

In the three-dimensional network aqueous gel according to the present invention, the water-soluble polymer comprises 10-30 wt % of sodium alginate, 10-30 wt % of polyvinylpyrrolidone, and 10-40 wt % of sodium carboxymethyl cellulose, calculated on the basis of 100 wt % of total weight percentage of the water-soluble polymer.

In the three-dimensional network aqueous gel according to the present invention, the three-dimensional network structure comprises a plurality of gel pores formed therein, and the gel pores have a diameter of 16-18 μm.

To achieve another objective, the present invention provides a manufacturing method of a three-dimensional network aqueous gel, and the method comprises: adding a water-soluble polymer into a solvent and uniformly mixing to form a homogeneous solution, wherein the water-soluble polymer comprises sodium carboxymethyl cellulose; subjecting the water-soluble polymer to a hydrolysis reaction to generate nanometer-scale water-soluble polymer particles and form a sol; under a pressure of 25-760 torr, subjecting the sol to vacuum conversion into a gel; subjecting the gel to a polycondensation reaction to have the nanometer-scale water-soluble polymer particles and the solvent interconnected to form a three-dimensional network structure; and subjecting the three-dimensional network structure to vacuum to form a three-dimensional network aqueous gel.

In the manufacturing method of a three-dimensional network aqueous gel according to the present invention, the water-soluble polymer further comprises sodium alginate, wherein sodium carboxymethyl cellulose and sodium alginate are interconnected with the solvent to form the three-dimensional network structure.

In the manufacturing method of a three-dimensional network aqueous gel according to the present invention, the water-soluble polymer further comprises polyvinylpyrrolidone, wherein sodium carboxymethyl cellulose, sodium alginate, and polyvinylpyrrolidone are interconnected with the solvent to form the three-dimensional network structure.

In the manufacturing method of a three-dimensional network aqueous gel according to the present invention, calculation made on the basis of 100 wt % of total weight percentage of the water-soluble polymer, the water-soluble polymer comprises 10-30 wt % of sodium alginate, 10-30 wt % of polyvinylpyrrolidone, and 10-40 wt % of sodium carboxymethyl cellulose.

In the manufacturing method of a three-dimensional network aqueous gel according to the present invention, the three-dimensional network structure comprises a plurality of gel pores formed therein, and the gel pores have a diameter of 16-18 μm.

In the manufacturing method of a three-dimensional network aqueous gel according to the present invention, a temperature of the gel is controlled to be between 30-70 degrees, and a pressure is controlled to be between 50-70 millimeters of mercury to control a degree of swelling of the gel and a structure of the gel.

In the manufacturing method of a three-dimensional network aqueous gel according to the present invention, the solvent comprises pure water or an organic solvent.

In the three-dimensional network aqueous gel according to the present invention, high molecules of the aqueous gel polymer are interconnected to form the three-dimensional network structure, and the gel pores of the network are filled up with liquid. The gel in which crosslinking reaction occurs forms a covalent crosslinking network, and the characteristic behavior of the gel is swelling, but not dissolving. Next, the large number of hydrophilic groups of the aqueous gel may absorb and keep a large amount of water. Further, the three-dimensional network aqueous gel exhibits excellent property of biocompatibility and biodegradability, and is easy to synthesize, and shows excellent permeability for low molecule solutes. Further, the three-dimensional network aqueous gel may be subjected to a technical condition dependent operation to change the structure of the aqueous gel to regulate the degree of swelling of the aqueous gel.

### BRIEF DESCRIPTION OF THE DRAWINGS

FIG. 1 is a schematic view illustrating formation of a three-dimensional network aqueous gel according to the present invention;

FIG. 2 is a scanning electronic microscope (SEM) photograph of the three-dimensional network aqueous gel according to the present invention;

FIG. 3 is a flow chart showing a manufacturing method for formation of the three-dimensional network aqueous gel according to the present invention;

FIG. 4A shows a picture of an affected part of a bedsore patient taken before being applied with the three-dimensional network aqueous gel according to the present invention;

FIG. 4B shows a picture of the affected part of the bedsore patient taken after having been applied with the three-dimensional network aqueous gel according to the present invention for two (2) days;

FIG. 4C shows a picture of the affected part of the bedsore patient taken after having been applied with the three-dimensional network aqueous gel according to the present invention for seven (7) days;

FIG. 5A shows a picture of a diabetes foot taken before being applied with the three-dimensional network aqueous gel according to the present invention;

FIG. 5B shows a picture of the diabetes foot taken after having been applied with the three-dimensional network aqueous gel according to the present invention for two (2) days; and

FIG. 5C shows a picture of the diabetes foot taken after having been applied with the three-dimensional network aqueous gel according to the present invention for seven (7) days.

### DETAILED DESCRIPTION OF THE PREFERRED EMBODIMENT

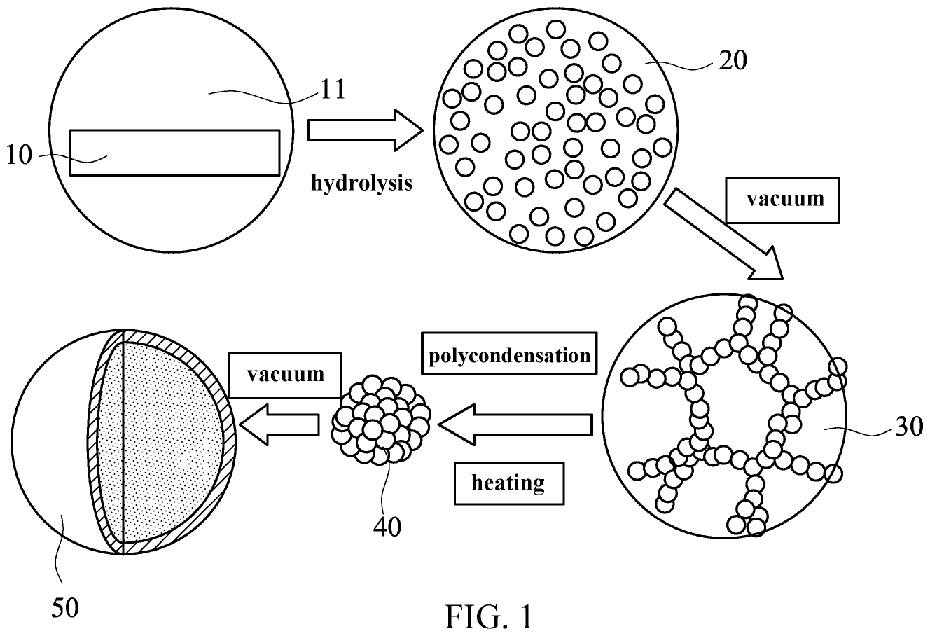

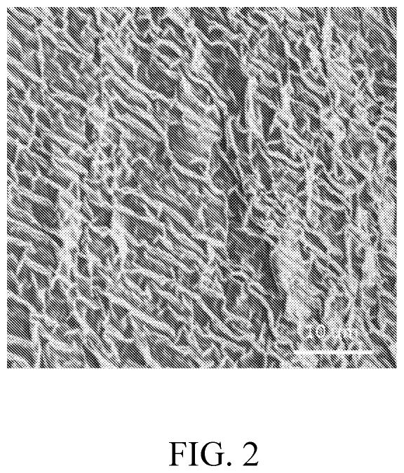

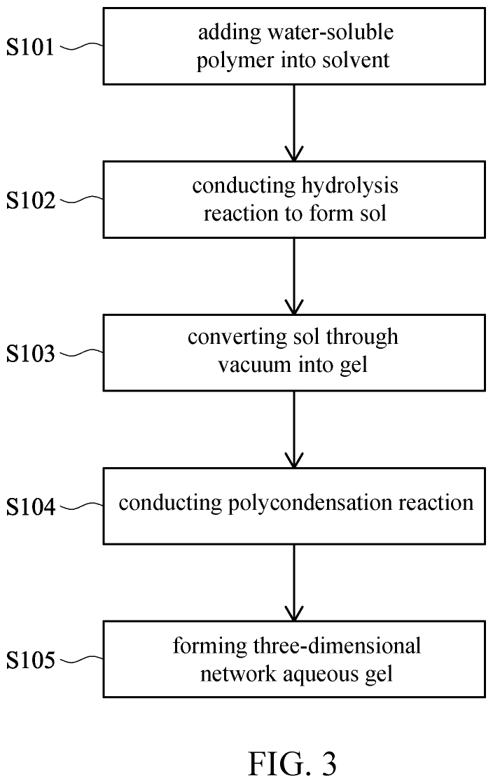

Referring to FIGS. 1-3, FIG. 1 is a schematic view illustrating formation of a three-dimensional network aqueous gel according to the present invention; FIG. 2 is a scanning electronic microscope (SEM) photograph of the three-dimensional network aqueous gel according to the present invention; and FIG. 3 is a flow chart showing a manufacturing method for formation of the three-dimensional network aqueous gel according to the present invention.

### Embodiment 1

As shown in FIGS. 1-3, the present invention provides a manufacturing method of a three-dimensional network aqueous gel. The method comprises: Step S101: adding a water-soluble polymer in water or an organic solvent and uniformly mixing together to form a homogeneous solution, wherein the water-soluble polymer comprises 80-95 wt % of sodium carboxymethyl cellulose based on the total weight percentage of 100 wt % of the water-soluble polymer. Step S102: subjecting the water-soluble polymer to a hydrolysis reaction to generate nanometer-scale water-soluble polymer particles and form a sol, the sol being a colloidal system having liquid characteristics having dispersed particles being solid or macromolecules, the dispersed particles having a size between 1-100 nm. Step S103: under a pressure of 25-760 torr, subjecting the sol to vacuum conversion into a gel, the gel being a colloidal system having solid characteristics, the dispersed substance forming a continuous network framework, framework gaps being filled with liquid or gas, the content of a dispersion phase in the gel being extremely low, generally between 1%-3%. Step S104: subjecting the gel to a polycondensation reaction and controlling a heating temperature between 30-70 degrees and a pressure between 50-70 millimeters of mercury, so as to induce interconnection between the nanometer-scale water-soluble polymer particles to form a three-dimensional network structure, wherein, specifically, the three-dimensional network structure provides the gel with an extremely high specific surface area. Step S105: subjecting the three-dimensional network structure to vacuum to form a three-dimensional network aqueous gel, wherein the three-dimensional network structure comprises a plurality of gel pores formed therein, and the gel pores have a diameter between 16-18 μm.

### Embodiment 2

EMBODIMENT 2 is generally similar to EMBODIMENT 1 in respect of the steps thereof, while a difference resides in that in EMBODIMENT 2, the water-soluble polymer further comprises sodium alginate, and based on 100 wt % of the total weight percentage of the water-soluble polymer, the water-soluble polymer comprises 10-30 wt % of sodium alginate and 70-90 wt % of sodium carboxymethyl cellulose.

### Embodiment 3

EMBODIMENT 3 is generally similar to EMBODIMENT 1 in respect of the steps thereof, while a difference resides in that in EMBODIMENT 3, the water-soluble polymer further comprises polyvinylpyrrolidone, and based on 100 wt % of the total weight percentage of the water-soluble polymer, the water-soluble polymer comprises 10-30 wt % of sodium alginate, 10-30 wt % of polyvinylpyrrolidone, and 10-40 wt % of sodium carboxymethyl cellulose.

### Swelling Degree Test

By controlling pressure and temperature, the structure of the three-dimensional network aqueous gel can be changed to regulate the degree of swelling of the three-dimensional network aqueous gel. The higher the temperature and pressure are, the smaller the diameter of the gel pores and the smaller the degree of swelling. The degree of swelling is calculated by means of the following formula: {(weight of three-dimensional network aqueous gel after swelling—weight of three-dimensional network aqueous gel before swelling)/weight of three-dimensional network aqueous gel before swelling×100}. In the swelling degree test, the water-soluble polymer of the three-dimensional network aqueous gel comprises 30 wt % of sodium alginate, 30 wt % of polyvinylpyrrolidone, and 40 wt % of sodium carboxymethyl cellulose, based on 100 wt % of the total weight percentage of the water-soluble polymer, and the result of the change of the gel pore and the degree of swelling of the three-dimensional network aqueous gel by means of temperature and pressure is shown in Table 1.

TABLE 1

| | | gel pore | degree of |
|---|---|---|---|
| temperature | pressure | diameter | swelling |
| (° C.) | (mmHg) | (μm) | (%) |
| 30 | 50 | 17.8 | 44.03% |
| 35 | 50 | 17.8 | 45.07% |
| 40 | 50 | 17.3 | 42.05% |
| 45 | 50 | 16.9 | 40.12% |
| 50 | 70 | 16.8 | 40.01% |
| 55 | 70 | 16.5 | 38.57% |
| 60 | 70 | 16.6 | 37.23% |
| 65 | 70 | 16.3 | 36.11% |
| 70 | 70 | 16.2 | 35.74% |

### Controlled Release Test

The water-soluble polymer of the three-dimensional network aqueous gel comprises 30 wt % of sodium alginate, 30 wt % of polyvinylpyrrolidone, and 40 wt % of sodium carboxymethyl cellulose, based on 100 wt % of the total weight percentage of the water-soluble polymer, and the controlled release time can be achieved by adjusting the three-dimensional network structure to provide an effect of controlled release. The controlled release time is estimated by using a transdermal absorption test apparatus, and pig skin is used as an artificial skin. The result is shown in the following Table 2.

TABLE 2

| | | gel pore | |
|---|---|---|---|
| temperature | pressure | diameter | controlled release |
| (° C.) | (mmHg) | (μm) | time (days) |
| 30 | 50 | 17.8 | 7 |
| 35 | 50 | 17.8 | 7 |
| 40 | 50 | 17.3 | 7.5 |
| 45 | 50 | 16.9 | 7 |
| 50 | 70 | 16.8 | 8 |
| 55 | 70 | 16.5 | 11 |
| 60 | 70 | 16.6 | 10.5 |
| 65 | 70 | 16.3 | 11 |
| 70 | 70 | 16.2 | 12 |

### Wound Healing Test

### Test Animal

The test uses male New Zealand rabbits of 8 weeks old, having a body weight of approximately 2000-2500 g. All the test animals are raised in an animal room having independent air conditioning with the room temperature being kept at 22° C. and relative humidity being kept at 45%, water and feed being sufficiently supplied. Before the test, the animals are given four weeks for adaption to the environment. Feeding environment, handling and all test procedures are in full compliance with “Guide for the Care and Use of Laboratory Animals” issued by (National Institutes of Health (NIH)).

### Formation of Skin Wound

The back part of the New Zealand rabbit is shaved, and sterilized with iodine tincture and 70% alcohol, and then, a skin wound of an area around 2 cm×2 cm is made on the back of the New Zealand rabbit by cutting with a surgical knife.

### Compositional Ingredients of Water-Soluble Polymer

TEST GROUP 1: The water-soluble polymer comprises, in the total weight percentage thereof, 25 wt % of sodium alginate, 25 wt % of polyvinylpyrrolidone, and 50 wt % of sodium carboxymethyl cellulose, calculated on the basis of 100 wt % of the total weight percentage of the water-soluble polymer.

TEST GROUP 2: The water-soluble polymer comprises, in the total weight percentage thereof, 30 wt % of sodium alginate, 30 wt % of polyvinylpyrrolidone, and 40 wt % of sodium carboxymethyl cellulose, calculated on the basis of 100 wt % of the total weight percentage of the water-soluble polymer.

COMPARISON GROUP 1: The water-soluble polymer comprises, in the total weight percentage thereof, 25 wt % of sodium alginate, 25 wt % of polyvinylpyrrolidone, and 50 wt % of sodium carboxymethyl cellulose, calculated on the basis of 100 wt % of the total weight percentage of the water-soluble polymer.

COMPARISON GROUP 2: The water-soluble polymer comprises, in the total weight percentage thereof, 30 wt % of sodium alginate, 30 wt % of polyvinylpyrrolidone, and 40 wt % of sodium carboxymethyl cellulose, calculated on the basis of 100 wt % of the total weight percentage of the water-soluble polymer.

### Application of Three-Dimensional Network Aqueous Gel

New Zealand rabbits are randomly divided into two test groups and two comparison groups, wherein the New Zealand rabbits of each of the groups are formed with a skin wound as described above, and then, the skin wounds of the New Zealand rabbits of TEST GROUPS 1 and 2 are applied with the three-dimensional network aqueous gels according to the above TEST GROUPS 1 and 2, and the wounds of the animals are covered with a polyurethane (PU) waterproof film to keep humid. The New Zealand rabbits of COMPARISON GROUPS are formed with a skin wound as described above, and then, the skin wounds of the New Zealand rabbits of COMPARISON GROUPS 1 and 2 are applied with the three-dimensional network aqueous gels according to the above COMPARISON GROUPS 1 and 2. The test is conducted for 14 days in total, and the wound areas of the New Zealand rabbits of each group are measured at the 2nd, 7th, and 14th days after the application of the dressing. The result is shown in the following Table 3.

TABLE 3

| | 2nd day after | 7th day after | 14th day after |
|---|---|---|---|
| | application | application | application |
| TEST GROUP 1 | 3.74 cm² | 3.35 cm² | 3.01 cm² |
| TEST GROUP 2 | 3.54 cm² | 3.09 cm² | 2.95 cm² |
| COMPARISON GROUP 1 | 3.96 cm² | 3.74 cm² | 3.61 cm² |
| COMPARISON GROUP 2 | 3.67 cm² | 3.44 cm² | 3.35 cm² |

A difference between TEST GROUP 1 and TEST GROUP 2 is the total weight percentage of the water-soluble polymer. The content of sodium carboxymethyl cellulose of TEST GROUP 2 is greater than that of TEST GROUP 1, and it is known from the above Table that a high content of sodium carboxymethyl cellulose increases the wound closure rate. Next, TEST GROUP 1 and TEST GROUP 2 cover a water-resistant film of polyurethane to keep the wound wet, while COMPARISON GROUP 1 and COMPARISON GROUP 2 do not apply a water-resistant film of polyurethane to cover the wounds, and it is known from the above Table that the healing rate can be much faster if the wounds are kept in a humid or wet environment. In summary of the above, wet healing of a wound provides the following advantage. Firstly, it is advantageous for dissolution of necrotic tissues and fibrins, in a humid or wet environment, tissue plasmin contained in wound exudate may prompt the dissolution and absorption of the necrotic tissue. Further, it keeps the wound site at a fixed temperature, accelerate division of cells, prompt wound healing, develop localized wetting and reduce formation of scabs, prevent mechanical damage to newly growing granulation tissues, reduce damages and pains for change of dressing, protect nerve endings at the wound to reduce pain. Further, in the closed wet-keeping environment, the dressing forms a barrier to reduce the chance of infection, and the slightly acidic environment in the closed condition could suppress growth of bacteria and help proliferation and functioning of white blood cells.

The three-dimensional network aqueous gel manufactured with the high-oxygen-content aqueous gel manufacturing method according to the present invention features containing of sodium carboxymethyl cellulose, polyvinylpyrrolidone, and sodium alginate and may form a colloidal body having high adhesion power, and can be made as a network polymer colloid that contains a great amount of water and have adhering property and excellent water absorbability. The colloid, when put in contact with a body surface, may induce repeated hydration reaction and exhibiting dual functions of supplying water toward the surface and absorbing exudate, by which bleeding and loss of body fluid can be controlled. Hydrophilic groups of sodium carboxymethyl cellulose, after absorbing water, becomes a gel form adhering to the wound site of blood vessel and swelling to form a gel layer to achieve wound hemostasis. Next, the high-oxygen-content aqueous gel forms a protective layer on the surface of a wound, which is colorless and clear, having a high moisture content, so as to keep the wound humid, prevent rubbing and irritating of the wound, not damaging the newly growing granulation tissues, and reducing secondary damage. Since sodium carboxymethyl cellulose contains acidic carboxyl group that is combinable with Fe²⁺ of hemoglobin to form a brown adhesive colloid block that achieves closure of endings of capillary vessels for stop bleeding. Further, the colloidal body also shows an effect of adhering and aggregating for platelets to thereby speed up blood clotting.

### Human Body Test

Referring to FIGS. 3A-3C, FIG. 3A shows a picture of an affected part of a bedsore patient taken before being applied with the three-dimensional network aqueous gel according to the present invention; FIG. 3B shows a picture of the affected part of the bedsore patient taken after having been applied with the three-dimensional network aqueous gel according to the present invention for two (2) days; and FIG. 3C shows a picture of the affected part of the bedsore patient taken after having been applied with the three-dimensional network aqueous gel according to the present invention for seven (7) days.

As shown in FIGS. 3A-3C, SUBJECT 1 of EMBODIMENT 1 is a bedsore patient. The wound of SUBJECT 1 is observable for fat tissues, and gives off odors, where granulation tissues, and edge curling, carrions, or scabs of the wound are observable, but fascia, muscle, tendon, ligament, cartilage, and bone are not observable. Application of the three-dimensional network aqueous gel according to the present invention is made to the affect part of the patient and making observation for the healing condition of the wound for 0 to 7 days. In the total weight percentage, the water-soluble polymer comprises 25 wt % of sodium alginate, 25 wt % of polyvinylpyrrolidone, and 50 wt % of sodium carboxymethyl cellulose, calculated on the basis of 100 wt % of the total weight percentage of the water-soluble polymer. After two days, blackening of skin has been improved, and the fat tissues in the wound become smaller after application for 7 days.

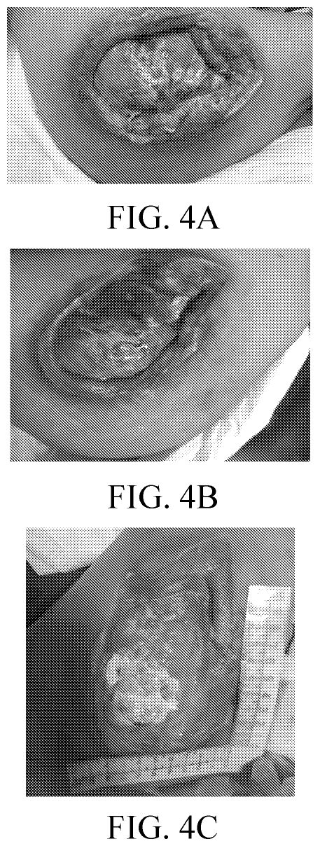

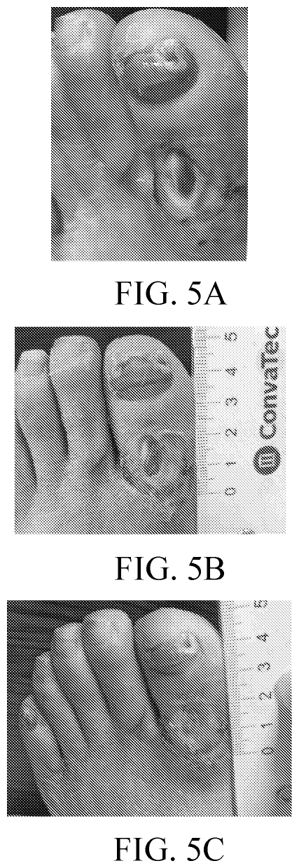

Referring to FIGS. 4A-4C, FIG. 4A shows a picture of a diabetes foot taken before being applied with the three-dimensional network aqueous gel according to the present invention; FIG. 4B shows a picture of the diabetes foot taken after having been applied with the three-dimensional network aqueous gel according to the present invention for two (2) days; and FIG. 4C shows a picture of the diabetes foot taken after having been applied with the three-dimensional network aqueous gel according to the present invention for seven (7) days.

As shown in FIGS. 4A-4C, SUBJECT 2 of EMBODIMENT 2 is a diabetes patient, and the foot tendon and ligament tissues have ulcerated and suppurative discharges and tissue necrosis have increased. After application of the three-dimensional network aqueous gel according to the present invention is made to the affect part of the patient and making observation for the healing condition of the wound for 0 to 7 days. In the total weight percentage, the water-soluble polymer comprises 25 wt % of sodium alginate, 25 wt % of polyvinylpyrrolidone, and 50 wt % of sodium carboxymethyl cellulose, calculated on the basis of 100 wt % of the total weight percentage of the water-soluble polymer. After two days, ulcer has been improved, and the wound is gradually healing after 7 days and the range of the wound shrinks.

In summary, the present invention provides a three-dimensional network aqueous gel, and a manufacturing method thereof. The three-dimensional network aqueous gel, upon application, forms a layer of extremely thin and invisible film on the surface of skin within 1 to 3 minutes. The film so formed, and the three-dimensional network structure contained in the film, can more effectively protect API (Active Pharmaceutical Ingredient) and achieve an effect of controlled release. The controlled release time can be varied to reach, maximally, up to 12 days by adjusting the three-dimensional network structure. Further, the three-dimensional network aqueous gel has effects of antiinflammation, smoothing wound, and preventing abnormal healing of wound, so as to achieve the purpose of speeding up wound healing and reducing pain of wound dressing change.

I claim:

1. A three-dimensional network aqueous gel, which is formed of a water-soluble polymer, the water-soluble polymer comprising sodium carboxymethyl cellulose, the water-soluble polymer being interconnected with a solvent to form a three-dimensional network structure.

2. The three-dimensional network aqueous gel according to claim 1, wherein the water-soluble polymer further comprises sodium alginate, wherein sodium carboxymethyl cellulose and sodium alginate are interconnected with the solvent to form the three-dimensional network structure.

3. The three-dimensional network aqueous gel according to claim 2, wherein the water-soluble polymer further comprises polyvinylpyrrolidone, wherein sodium carboxymethyl cellulose, sodium alginate, and polyvinylpyrrolidone are interconnected with the solvent to form the three-dimensional network structure.

4. The three-dimensional network aqueous gel according to claim 3, wherein the water-soluble polymer comprises 10-30 wt % of sodium alginate, 10-30 wt % of polyvinylpyrrolidone, and 10-40 wt % of sodium carboxymethyl cellulose, calculated on the basis of 100 wt % of total weight percentage of the water-soluble polymer.

5. The three-dimensional network aqueous gel according to claim 1, wherein the three-dimensional network structure comprises a plurality of gel pores formed therein, and the gel pores have a diameter of 16-18 m.

6. A manufacturing method of a three-dimensional network aqueous gel, the method comprising:
adding a water-soluble polymer into a solvent and uniformly mixing to form a homogeneous solution, wherein the water-soluble polymer comprises sodium carboxymethyl cellulose;
subjecting the water-soluble polymer to a hydrolysis reaction to generate nanometer-scale water-soluble polymer particles and form a sol;
under a pressure of 25-760 torr, subjecting the sol to vacuum conversion into a gel;
subjecting the gel to a polycondensation reaction to have the nanometer-scale water-soluble polymer particles and the solvent interconnected to form a three-dimensional network structure; and
subjecting the three-dimensional network structure to vacuum to form a three-dimensional network aqueous gel;
wherein a temperature of the gel is controlled to be between 30-70 degrees, and a pressure is controlled to be between 50-70 millimeters of mercury to control a degree of swelling of the gel and a structure of the gel.

7. The manufacturing method according to claim 6, wherein the water-soluble polymer further comprises sodium alginate, wherein sodium carboxymethyl cellulose and sodium alginate are interconnected with the solvent to form the three-dimensional network structure.

8. The manufacturing method according to claim 7, wherein the water-soluble polymer further comprises polyvinylpyrrolidone, wherein sodium carboxymethyl cellulose, sodium alginate, and polyvinylpyrrolidone are interconnected with the solvent to form the three-dimensional network structure.

9. The manufacturing method according to claim 8, wherein calculation made on the basis of 100 wt % of total weight percentage of the water-soluble polymer, the water-soluble polymer comprises 10-30 wt % of sodium alginate, 10-30 wt % of polyvinylpyrrolidone, and 10-40 wt % of sodium carboxymethyl cellulose.

10. The manufacturing method according to claim 5, wherein the three-dimensional network structure comprises a plurality of gel pores formed therein, and the gel pores have a diameter of 16-18 μm.
